(19) United States
(12) Patent Application Publication
(10) Pub. No.: US 2018/0028470 A1
(43) Pub. Date: Feb. 1, 2018

(54) CURCUMIN-BASED PHARMACEUTICAL COMPOSITIONS AND METHODS FOR FABRICATING THEREOF

(71) Applicant: IMPRIMIS PHARMACEUTICALS, INC., San Diego, CA (US)

(72) Inventors: Dennis Elias Saadeh, Irvine, CA (US); Nadia Ibrahim, San Diego, CA (US)

(21) Appl. No.: 15/653,769

(22) Filed: Jul. 19, 2017

Related U.S. Application Data
(60) Provisional application No. 62/366,807, filed on Jul. 26, 2016.

(51) Int. Cl. A61K 31/12; A61K 47/26; A61K 47/34; A61K 47/44; A61K 9/10; A61K 9/00; A61K 47/40

(52) U.S. Cl. CPC A61K 31/12; A61K 9/0019; A61K 47/26; A61K 47/40; A61K 47/44; A61K 9/10; A61K 47/34

(57) ABSTRACT

Pharmaceutical compositions are described, the compositions consisting essentially of aqueous emulsions of a therapeutically effective quantity of a diarylheptanoid compound (such as curcumin). Methods for fabricating the compositions and using them are also described.

### CROSS-REFERENCE TO THE RELATED APPLICATION

This application claims the benefit of priority under 35 U.S.C. § 119(e) of U.S. Provisional Application No. 62/366,807, filed Jul. 26, 2016, the entire content of which is incorporated herein by reference.

### FIELD OF THE INVENTION

The present invention relates generally to the field of pharmaceuticals and more specifically to pharmaceutical compositions that include as an active component a therapeutically effective quantity of a diarylheptanoid compound (such as curcumin), and to methods of preparing and using such compositions.

### BACKGROUND

Turmeric and its active ingredient, curcumin, has been used for a long time to treat a variety of diseases and conditions. It is an anti-inflammatory substance and may help in treating neoplasia (i.e., various cancers), digestive problems, liver problems, skin diseases, wounds, indigestion, dyspepsia, ulcers, colitis, heart disease, etc. According to some theories, the health benefits attributable to curcumin stem from it being a powerful anti-oxidant. It is, of course, well known that anti-oxidants scavenge free radicals in the body, and free radicals are capable of damaging cell membranes, tampering with DNA, and even causing cell death. Antioxidants can fight free radicals and may reduce or even help prevent some of the damage they cause. In addition, curcumin is believed to lower the levels of certain enzymes in the body that cause inflammation. It is also theorized that curcumin stops platelets from clumping together to form blood clots.

While the exact mechanism by which curcumin is able to impart valuable health benefits remains unclear, the overall positive effect is quite pronounced in some cases. Indeed, a number of studies have demonstrated that curcumin has anti-proliferative properties, perhaps by modulating multiple cell-signaling pathways. Such an effect has been shown for various kinds of cancers, including pancreatic, hepatic, colorectal, ovarian, breast, lung, prostate, and head and neck.

In spite of a significant body of evidence in favor of using turmeric (and, consequently, curcumin contained therein), it has been determined that it may not work as well in many cases. One serious drawback of turmeric is its poor bioavailability, probably because of its poor solubility in water.

It is, therefore, desirable to have curcumin-based pharmaceutical compositions that are free from drawbacks and deficiencies. This patent specification discloses such pharmaceutical compositions that can achieve such positive patient outcomes, and methods of fabricating and administering the same.

### SUMMARY

According to one embodiment of the invention, a pharmaceutical composition formulated as a colloidal emulsion is provided, the composition consisting of a dispersed phase consisting of particles consisting of a therapeutically effective quantity of at least one diarylheptanoid compound, or derivatives or analogs thereof, and a dispersion medium consisting of a therapeutically effective quantity of pharmaceutically acceptable solubilizing and suspending agent selected from the group comprising at least one vegetable oil, at least one non-ionic polyoxyethlene-polyoxypropylene block copolymer, optionally at least one additional solubilizing and suspending agent, and a pharmaceutically acceptable carrier, wherein the dispersed phase is dispersed within the dispersion medium.

According to another embodiment of the invention, the diarylheptanoid compound is a linear diarylheptanoid, such as curcumin.

According to yet another embodiment of the invention, the polyoxyethlene-polyoxypropylene block copolymers that is a part of the dispersion medium is a copolymer of Poloxamer® or Pluronic® family, e.g., Poloxamer 407® or Pluronic® L64.

According to another embodiment of the invention, the additional solubilizing and suspending agent, if used, is any of water-soluble derivatives of cellulose (e.g., carboxymethyl cellulose, methyl cellulose, hydroxyethyl cellulose, or hydroxypropyl cellulose), non-cross-linked or partially cross-linked polyacrylates, polyoxyethylene sorbitan monolaurates, polyoxyethylene sorbitan monopalmitates, polyoxyethylene sorbitan monostearates, and polyoxyethylene sorbitan monooleates.

According to other embodiments of the invention, the pharmaceutical compositions described herein may be administered to a mammalian subject in need of such treatment, to treat various neoplastic diseases and maladies (i.e., cancer).

### DETAILED DESCRIPTION

### A. TERMS AND DEFINITIONS

Unless specific definitions are provided, the nomenclatures utilized in connection with, and the laboratory procedures and techniques of analytical chemistry, synthetic organic and inorganic chemistry described herein, are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, formulating compositions and testing them. The foregoing techniques and procedures can be generally performed according to conventional methods well known in the art.

It is to be understood that both the foregoing general description and the following detailed description are exemplary and explanatory only and are not restrictive of the invention claimed. As used herein, the use of the singular includes the plural unless specifically stated otherwise. The section headings used herein are for organizational purposes only and are not to be construed as limiting the subject matter described.

As used herein, “or” means “and/or” unless stated otherwise. Furthermore, use of the term “including” as well as other forms, such as “includes,” and “included,” is not limiting.

“About” as used herein means that a number referred to as “about” comprises the recited number plus or minus 1-10% of that recited number. For example, “about” 100 degrees can mean 95-105 degrees or as few as 99-101 degrees depending on the context. Whenever it appears herein, a numerical range such as “1 to 20” refers to each integer in the given range; i.e., meaning only 1, only 2, only 3, etc., up to and including only 20.

The term “pharmaceutical composition” is defined as a chemical or a biological compound or substance, or a mixture or combination of two or more such compounds or substances, intended for use in the medical diagnosis, cure, treatment, or prevention of disease or pathology.

The term “emulsion” is defined for the purposes of the present application as a two-phase liquid-in-liquid dispersion system having a first phase and a second phase. In other words, an IUPAC definition of “emulsion” is adopted herein, according to which it is a fluid system in which liquid droplets are dispersed in another liquid. It is further specifically provided that dispersion systems having three, four or more phases are not within the meaning of “emulsion” for the purposes of the instant application.

Furthermore, the above mentioned first phase of the emulsion consists of a multitude of liquid particles and is designated and defined as the dispersed phase, and the above mentioned second phase of the emulsion is also a liquid and is designated and defined as the dispersion medium, or, interchangeably and synonymously, the continuous phase.

Furthermore, the above mentioned dispersed phase is dispersed in the above mentioned dispersion medium, and the term “dispersed” is defined as meaning that the dispersed phase is statistically evenly distributed within the continuous phase throughout the entire volume of the emulsion, with no statistically meaningful deviations in the concentrations of the dispersed phase in different portions of the emulsion.

The term “castor oil” refers to a compound that is based on ricinoleic acid, an unsaturated, 18-carbon fatty acid having a hydroxyl functional group on the 12^{th} carbon (IUPAC, 12-hydroxyoctadec-9-enoic acid). Those having ordinary skill in the art will understand that castor oil has a very complex chemical structure and comprises a mixture of triglycerides of ricinoleic acid (about 80%) plus triglycerides of linoleic (IUPAC, 9,12-octadecadienoic) and oleic (IUPAC, octadec-9-enoic) acids (about 20% combined). The principal triglyceride component has a simplified chemical structure shown below:

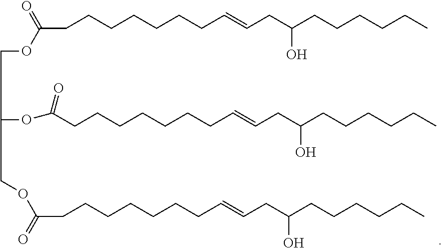 [SMILES: CCCCCCC(O)C/C=C/CCCCCCCC(=O)OCC(COC(=O)CCCCCCC/C=C/CC(O)CCCCCC)OC(=O)CCCCCCC/C=C/CC(O)CCCCCC]

While the principal compound is based on the unsaturated ricinoleic acid, as stated and shown above, some compounds in the mixture may include some partially hydrogenated fragments where the double bond at the 9^{th} carbon is saturated.

The term “PEGylated” refers to vegetable oils having a quantity of poly(ethylene glycol) (PEG) covalently or non-covalently attached to the oil molecules. “PEGylation” means a process of obtaining such PEGylated products

The term “carrier” refers to a substance that serves as a vehicle for improving the efficiency of delivery and the effectiveness of a pharmaceutical composition.

The term “solubilizing agent” for the purposes of the instant application refers broadly to chemical compounds that improve the process of incorporating the solubilizate (i.e., active components described herein) into micelles; in other words, the presence of a solubilizing agent makes the process of solubilization faster, easier, and/or more complete compared with compositions without it.

The term “suspending agent” for the purposes of the instant application refers broadly to chemical compounds that help active pharmaceutical ingredients stay suspended in the formulation and prevents and/or reduces the phase separation of two-phase dispersion systems described herein.

The term “therapeutically effective amount” is defined as the amount of the compound or pharmaceutical composition that will elicit the biological or medical response of a tissue, system, animal or human that is being sought by the researcher, medical doctor or other clinician.

The term “pharmaceutically acceptable” is defined as a carrier, whether diluent or excipient, that is compatible with the other ingredients of the formulation and not deleterious to the recipient thereof.

The terms “administration of a composition” or “administering a composition” are defined to include an act of providing a compound of the invention or pharmaceutical composition to the subject in need of treatment.

### B. EMBODIMENTS OF THE INVENTION

According to embodiments of the present invention, pharmaceutical compositions intended to prevent and/or treat various diseases and maladies, such as neoplasms, are provided. The compositions are in the form of colloidal emulsions. The emulsions include a dispersion medium (i.e., the continuous phase), a dispersed phase that is dispersed within the dispersion medium, and a pharmaceutically acceptable carrier. The dispersed phase includes particles of a therapeutically effective quantity of at least one active component, a diarylheptanoid compound, which may be linear, or derivatives or analogs thereof. The dispersion medium includes at least two, and may optionally include more than two, solubilizing and suspending agents.

As mentioned above, one diarylheptanoid compound or a combination of several such compounds can be used to form an active component. One example of a linear diarylheptanoid compound that may be used is a curcumin, which is has the IUPAC name 1,7-bis(4-hydroxy-3-methoxyphenyl)-1,6-heptadiene-3,5-dione, a compound having the following chemical structure:

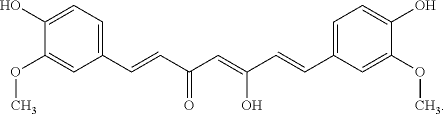 [SMILES: COc1cc(/C=C/C(=O)/C=C(O)/C=C/c2ccc(O)c(OC)c2)ccc1O]

An enol form of curcumin is illustrated above, but a keto form (not shown) may be also used instead; any tautomeric mixture of the keto and enol forms, whether naturally occurring or otherwise, may be used as well.

Other acceptable linear diarylheptanoid compounds that may be used in addition to, or instead of, curcumin, include, desmethoxycurcumin, bis-desmethoxycurcumin, and combinations thereof. Those having ordinary skill in the art may use other diarylheptanoid(s) instead of, or in combination with, the above-named heparinoids, if desired.

The mass concentration of curcumin in the composition may be between about 1.0% and about 2.0%, or between about 5 mg/ml and about 50 mg/ml, such as between about 10 mg/ml and about 20 mg/ml.

According to all the embodiments, at least three distinct components are always present in the dispersion medium. These required compounds include:

(a) at least one vegetable oil, such as castor oil, soybean oil, coconut oil, avocado oil, olive oil or almond oil;

(b) as the first solubilizing and suspending agent, at least one non-ionic polyoxyethlene-polyoxypropylene block copolymer having the following general structure:

HO—(CH₂—CH₂—O)ₓ—(C₃H₆—O)_{y}—(CH₂—CH₂—O)ₓ—H,

wherein in the chemical structure above x is an integer having the value of at least 8 and y is an integer having the value of at least 38; and

(c) a pharmaceutically acceptable carrier such as de-ionized and purified water.

According to all the embodiments, the dispersion medium may contain between about 10.0 mass % and 30.0 mass %, such as between about 15.0 mass % and 20.0 about mass %, for example, about 17.0 mass % of vegetable oil(s), and between about 0.5 mass % and about 10.0 mass % such as between about 2.0 mass % and about 8.0 mass %, for example, about 2.0 mass % of the non-ionic polyoxyethlene-polyoxypropylene block copolymer. The balance of the dispersion medium comprises water and optionally at least one additional solubilizing and stabilizing agent, if desired.

The above-mentioned vegetable oil(s) may be optionally partially PEGylated. For example, in a PEGylated castor oil the hydroxyl groups of the castor oil triglyceride are ethoxylated with ethylene oxide to form polyethylene glycol ethers. Those having ordinary skill in the art may synthesize PEGylated products by PEGylation of vegetable oil(s) according to known techniques and methods, so that the PEGylated product comprise between about 20.0 and about 50.0 moles of PEG of monomeric ethylene oxide per mole of the vegetable oil.

If desired, commercially available PEGylated oil(s) can be utilized. One specific non-limiting example of a commercially available PEGylated oil is PEG-40 castor oil containing about 40 moles of monomeric ethylene oxide per mole of castor oil. Other examples of commercially available PEGylated oils that those having ordinary skill in the art may find acceptable for the purposes of the instant application include PEG-30, PEG-33, PEG-35, or PEG-36 castor oil (containing 30, 33, 35, or 36 moles of monomeric ethylene oxide, respectively per mole of castor oil). All above mentioned PEGylated castor oils are available from BASF Corp. (Florham Park, N.J.) under several trade names such as Cremophor® or Kolliphor®.

Polyoxyethlene-polyoxypropylene block copolymer(s) that can be used as the first solubilizing and stabilizing agent in the pharmaceutical compositions of the instant invention may be those belonging to the Pluronic® or Poloxamer® families, chemically, poly(ethylene glycol)-block-poly(propylene glycol)-block-poly(ethylene glycol), both available from BASF Corp. and from several other vendors and having the following general chemical structure

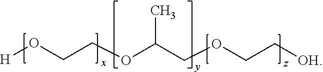

One non-limiting example of a specific non-ionic polyoxyethlene-polyoxypropylene block copolymer that can be used as the first solubilizing and stabilizing agent in the pharmaceutical compositions of the instant invention is the product known under the trade name Pluronic® L64, which is described by the chemical structure above, with the molecular weight of the polyoxypropylene portion of about 1,750 Daltons, about a 40% polyoxyethylene content (mass), and the average overall molecular weight of about 2,900 Daltons.

Another non-limiting example of a specific non-ionic polyoxyethlene-polyoxypropylene block copolymer that can be used as the first solubilizing and stabilizing agent in the pharmaceutical compositions of the instant invention is the product known under the trade name Poloxamer 407® (also known as Pluronic® F127), which is also described by the chemical structure above, with the molecular weight of the polyoxypropylene portion of about 4,000 Daltons, about a 70% polyoxyethylene content (mass), the overall molecular weight of between about 9,840 Daltons and about 14,600 Daltons.

As stated above, some embodiments provide for an optional inclusion of additional solubilizing and suspending agent(s) into the dispersion medium. Some non-limiting examples of such additional solubilizing and suspending agent(s) include water-soluble derivative of cellulose, optionally partially cross-linked polyacrylates, polyoxyethylene sorbitan monolaurates, polyoxyethylene sorbitan monopalmitates, polyoxyethylene sorbitan monostearates, polyoxyethylene sorbitan monooleates (e.g., members of Polysorbate® family of products).

Those having ordinary skill in the art will realize that other additional solubilizing and suspending agent(s) may be used if desired, and will select such supplemental solubilizing and suspending agent(s), as well as to choose the quantity thereof.

More specifically, suitable water-soluble derivatives of cellulose that may be used as additional solubilizing and suspending agent(s) include, without limitations, carboxymethyl cellulose, methyl cellulose, hydroxyethyl cellulose, and hydroxypropyl cellulose available, among other sources, from The Dow Chemical Company of Midland, Mich. Examples of acceptable water-soluble, partially cross-linked, polyacrylates that may be used include, without limitations, such as polymers of the Carbopol® family available from The Lubrizol Corporation of Wickliffe, Ohio. Typically, the cross-linking agents that may be used to cross-link such polyacrylates are allyl sucrose or allyl pentaerythritol.

Suitable products of Polysorbate® family (i.e., ethoxylated sorbitan esterified with fatty acids) that may be used include, without limitation, polyoxyethylene sorbitan monolaurates, polyoxyethylene sorbitan monopalmitates, polyoxyethylene sorbitan monostearates, or polyoxyethylene sorbitan monooleates, some of which are also known as Tween® products, such as Polysorbate® 80) can be used as the second solubilizing and stabilizing agent. Such products are available from Croda Americas, L.L.C. of Wilmington, Del. or from Sigma-Aldrich Corp., among other suppliers making these products available.

One typical product of the latter family that can be used is Polysorbate® 80 (alternatively, Tween® 80) (chemically, polyoxyethylene (20) sorbitan monooleate, also known as sorbitan mono-9-octadecenoate poly(oxy-1,2-ethanediyl), i.e., a product of polycondensation of polyethoxylated sorbitan and oleic acid having 20 units derived from ethylene glycol), a nonionic surfactant and emulsifier having the structure,

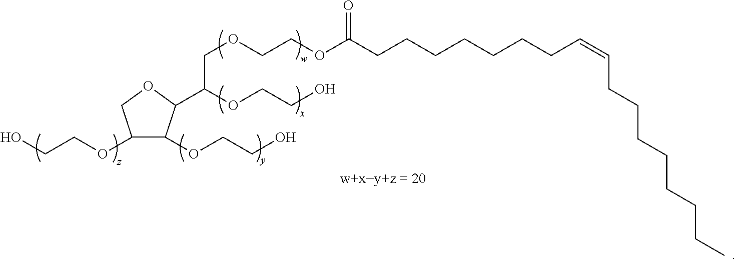

According to embodiments of the present application, the pharmaceutical compositions described herein are formulated as stable two-phase emulsions as defined above. More specifically, according to these embodiments, the emulsions consist of two phases, i.e., the dispersed phase that is dispersed within the dispersion medium. The dispersed phase consists of particles consisting of a therapeutically effective quantity of the pharmaceutically active component, i.e., a diarylheptanoid compound, or derivatives or analogs thereof. In some embodiments, no compounds other than diarylheptanoid compounds described hereinabove are present within the particles that form the dispersed phase.

According to such embodiments, the dispersion medium is a liquid that includes all other compounds that are present in the pharmaceutical compositions described in the application. The application envisions no embodiment where diarylheptanoid compounds can be used outside the dispersed phase such as being a part of the dispersion medium.

A brief summary of various exemplary, non-limiting products that can be used to form the dispersion medium of compositions of the present invention (including those mentioned and some additional products), as well as their classification, is also presented in Table 1.

TABLE 1
Some Common Products Useful in Dispersion Medium of Compositions

| Classification | Exemplary Products |
|---|---|
| Polyglycolyzed Glycerides | PEG-8 glyceryl capralate/caprate (LABRASOL ®) |
| | PEG-32 glyceryl laurate (GELUCIRE ® 44/14) |
| | PEG-32 glyceryl palmito stearate (GELUCIRE ® 50/13) |
| Polyoxyethylene Sorbitan Fatty | Polyoxyethylene 20 sorbitan monolaurate (TWEEN ® 20) |
| Acid Esters | Polyoxyethylene 20 sorbitan stearate (TWEEN ® 60) |
| | Polyoxyethylene 20 sorbitan monooleate (TWEEN ® 80) |
| Sorbitan Fatty Acid Esters | Sorbitan monolaurate (SPAN ® 20) |
| | Sorbitan monostearate (SPAN ® 60) |
| | Sorbitan monooleate (SPAN ® 80) |
| Polyoxyethylene Castor Oil | Polyoxyl 35 castor oil (Cremophor ® EL) |
| Derivatives | Polyoxyl 40 hydrogenated castor oil (Cremophor ® RH 40) |
| Polyethylene Glycol-Based | D-α-tocopherol polyethylene glycol-1000 succinate (TPGS) |
| Derivatives of Vitamin E | |
| Phospholipids and PEG | Lecithin |
| Phospholipids | Modified lecithin |
| Cyclic Oligosaccharides | Hydroxypropyl β-cyclodextrin |

In other embodiments, in addition to a diarylheptanoid compound, or derivatives or analogs thereof, the dispersed phase may optionally contain other compounds, such as stabilizers, anti-oxidants, chelating agents, etc.

According to further embodiments, methods for fabricating the above-described pharmaceutical compositions are provided. A one-batch formulation method may be used, where the components of the pharmaceutical formulation can be combined in single container; the components may be added to the container simultaneously or consecutively.

In one exemplary, non-limiting procedure, the process of preparing the pharmaceutical compositions described hereinabove may commence by fabricating a dispersion phase and proceeding in the specified order of steps. First, predetermined quantities of vegetable oil(s) (e.g., PEG-40 castor oil or Polyoxyl 35 castor oil), non-ionic polyoxyethlene-polyoxypropylene block copolymer (e.g., Poloxamer® 407), and additional solubilizing and suspending agent(s) (if used) are thoroughly mixed and heated to a temperature between about 50° C. and about 70° C. for about 20 to 30 minutes. Next, a premeasured quantity of curcumin is added to the dispersion phase while the latter is being stirred and maintained at the temperature in the 50-70° C. range. The stirring at this temperature is continued for about 30 minutes. Immediately thereafter, water and other components, if used (e.g., stabilizers, chelating agents, etc.) are slowly added while the stirring and heating are maintained. Finally, the composition so obtained is subjected to a shear force in a homogenizer for about 30 minutes, and cooled. As a result, a stable colloidal emulsion is obtained.

While the composition can be obtained in a different way by following a different sequence of steps, it is believed that those having ordinary skill in the art will find that it is beneficial to follow the manufacturing procedure described above as the final product is expected to have both superior stability and salubrious properties. In particular, without elaborating on the mechanism of the process, it is believed that the heating protocol described above ensures a better quality in terms of stability and potency compared with the old method of making Poloxamer-based colloidal systems (i.e., heating only until flakes disappear).

Pharmaceutical compositions prepared as described above can be used to treat, prevent or alleviate neoplastic diseases and conditions, including benign, precancerous and malignant tumors as well as non-solid tumors such as lymphomas and leukemia.

Pharmaceutical formulations described herein can be typically delivered via injections, e.g., intravenously, intramuscularly or subcutaneously. An ordinarily skilled physician may prescribe delivery by any other acceptable method if so desired and indicated.

It will be understood by those having ordinary skill in the art that the specific dose level and frequency of dosage for any particular patient may be varied and will depend upon a variety of factors including the activity of the specific compound employed, the metabolic stability and length of action of that compound, the age, body weight, general health, gender, diet, and the severity of the particular disease or condition being treated.

In additional embodiments, pharmaceutical kits are provided. The kit includes a sealed container approved for the storage of pharmaceutical compositions, the container containing one of the above-described pharmaceutical compositions. An instruction for the use of the composition and the information about the composition are to be included in the kit.

The following example is provided to further elucidate the advantages and features of the present invention, but are not intended to limit the scope of the invention. The example is for the illustrative purposes only. USP pharmaceutical grade products were used in preparing the formulations described below.

### C. EXAMPLES

### Example 1

### Preparing a Pharmaceutical Composition

A pharmaceutical composition was prepared as described below. The following products were used in the amounts specified:

(a) about 1.05 g of 95% curcumin powder;

(b) about 4.0 mL of glycerin;

(c) about 4.0 mL of Polysorbate® 80;

(d) about 15 mL of PEG-40 castor oil;

(e) about 0.5 mL of Pluronic® L64;

(f) about 0.1 g of ededate disodium dehydrate powder;

(g) about 0.1 g of vitamin E acetate;

(h) about 2.0 mL of benzyl alcohol; and

(i) about 100.0 mL of water, sterile for injection.

To a beaker equipped with a stir bar, there were added glycerin and Polysorbate® 80 and the mixture was heated to, and maintained at, a temperature between about 50° C. and 70° C. PEG-40 castor oil and Pluronic® L64 were added, with continued stirring and while maintaining the temperature within the same range. Curcumin power was then added slowly and the mixing was continued for about 30 minutes, at the same temperature.

About 50% of water was then slowly added, followed by adding benzyl alcohol, ededate disodium dehydrate powder, vitamin E, while continuing mixing for about 30 minutes, at the same temperature. The mixture was then homogenized for about 30 minutes, using the shear force of a standard homogenizer, and the balance of water was added. The final product was then warm-filtered through the 0.22 μM filter and placed into sterile amber vials, followed by testing for potency and sterility using standard tests, both of which were passed.

Although the invention has been described with reference to the above examples, it will be understood that modifications and variations are encompassed within the spirit and scope of the invention. Accordingly, the invention is limited only by the following claims.

What is claimed is:

1. A pharmaceutical composition formulated as a colloidal emulsion that consists of:
(a) a dispersed phase consisting of particles consisting of a therapeutically effective quantity of at least one diarylheptanoid compound, or derivatives or analogs thereof; and
(b) a dispersion medium consisting of:
(b 1) at least one vegetable oil;
(b2) at least one pharmaceutically acceptable solubilizing and suspending agent selected from the group consisting of non-ionic polyoxyethlene-polyoxypropylene block copolymers;
(b3) optionally at least one additional solubilizing and suspending agent; and
(b4) a pharmaceutically acceptable carrier,
wherein the dispersed phase is dispersed within the dispersion medium.

2. The pharmaceutical composition of claim 1, wherein the additional solubilizing and suspending agent is present in the dispersion medium, the additional solubilizing and suspending agent being selected from the group consisting of a water-soluble derivative of cellulose, optionally partially cross-linked polyacrylates, polyoxyethylene sorbitan monolaurates, polyoxyethylene sorbitan monopalmitates, polyoxyethylene sorbitan monostearates, polyoxyethylene sorbitan monooleates, cyclodextrin and derivatives thereof.

3. The pharmaceutical composition of claim 1, wherein the diarylheptanoid compound is a linear diarylheptanoid.

4. The pharmaceutical composition of claim 3, wherein the linear diarylheptanoid is selected from the group consisting of curcumin, desmethoxycurcumin, bis-desmethoxycurcumin, and combinations thereof.

5. The pharmaceutical composition of claim 3, wherein the linear diarylheptanoid has the chemical structure A:

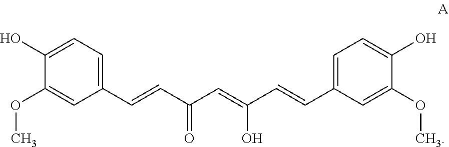 [SMILES: COc1cc(/C=C/C(=O)/C=C(O)/C=C/c2ccc(O)c(OC)c2)ccc1O]

6. The pharmaceutical composition of claim 4, wherein the linear diarylheptanoid is curcumin.

7. The pharmaceutical composition of claim 2, wherein the water-soluble derivative of cellulose is selected from the group consisting of carboxymethyl cellulose, methyl cellulose, hydroxyethyl cellulose, and hydroxypropyl cellulose.

8. The pharmaceutical composition of claim 2, wherein the second solubilizing and suspending agent is polyoxyethylene (20) sorbitan monooleate.

9. The pharmaceutical composition of claim 2, wherein the second solubilizing and suspending agent is hydroxypropyl β-cyclodextrin.

10. The pharmaceutical composition of claim 1, wherein the mass concentration of curcumin in the composition is between about 1.0% and about 2.0%.

11. The pharmaceutical composition of claim 1, wherein the vegetable oil is selected from the group consisting of castor oil, soybean oil, coconut oil, avocado oil, olive oil, and almond oil.

12. The pharmaceutical composition of claim 1, wherein the vegetable oil is a PEGylated castor oil.

13. The pharmaceutical composition of claim 1, wherein the non-ionic polyoxyethlene-polyoxypropylene block copolymer is poly(ethylene glycol)-block-poly(propylene glycol)-block-poly(ethylene glycol).

14. A method for treating, preventing or alleviating a disease, condition, syndrome, symptom, pathology, or malady in a mammalian subject in need of such treatment comprising delivery to the subject the composition of claim 1.

15. The method of claim 14, wherein the disease being treated is neoplasia.

16. A method for fabricating a pharmaceutical composition of claim 1, the method comprising:
(a) combining at least one vegetable oil, at least one non-ionic polyoxyethlene-polyoxypropylene block copolymer, and optionally at least one additional solubilizing and suspending agent to form a first mixture, followed by
(b) while stirring, heating the first mixture to a temperature between about 50° C. and about 70° C. for about 20 to 30 minutes, followed by
(c) adding to the first mixture a therapeutically effective quantity of a diarylheptanoid compound, or derivatives or analogs thereof to form a second mixture, followed by
(d) maintaining the second mixture at a temperature between about 50° C. and about 70° C. for about 30 minutes, while stirring, followed by
(e) adding a pharmaceutically acceptable carrier while stirring for about 30 minutes, while maintaining the second mixture at a temperature between about 50° C. and about 70° C. to form a third mixture, followed by
(f) subjecting the third mixture to a shear force in a homogenizer for about 30 minutes,
to obtain the pharmaceutical composition thereby.

17. The method of claim 16, wherein the diarylheptanoid compound is a linear diarylheptanoid selected from the group consisting of curcumin, desmethoxycurcumin, bis-desmethoxycurcumin, and combinations thereof.

18. The method of claim 16, wherein the linear diarylheptanoid is curcumin.

19. The method of claim 16, wherein the vegetable oil is selected from the group consisting of castor oil, soybean oil, coconut oil, avocado oil, olive oil, and almond oil.

20. The method of claim 19, wherein the vegetable oil is a PEGylated castor oil.

21. The method of claim 16, wherein the non-ionic polyoxyethlene-polyoxypropylene block copolymer is poly(ethylene glycol)-block-poly(propylene glycol)-block-poly(ethylene glycol).
